(19) United States
(12) Patent Application Publication
(10) Pub. No.: US 2005/0282174 A1
(43) Pub. Date: Dec. 22, 2005

(54) Methods and systems for selecting nucleic acid probes for microarrays

(76) Inventors: Peter G. Webb, Menlo Park, CA (US); Stephanie B. Fulmer-Smentek, Sunnyvale, CA (US); Patrick J. Collins, San Francisco, CA (US); Karen W. Shannon, Los Gatos, CA (US); Jing Gao, Santa Clara, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/871,303

(22) Filed: Jun. 19, 2004

(51) Int. Cl. C12Q 1/68

(52) U.S. Cl. 435/6

(57) ABSTRACT

Methods and systems for identifying and selecting nucleic acid probes for detecting a target with a nucleic acid probe array or microarray, comprising selecting a plurality of candidate probes, forming a plurality of clusters from the plurality of candidate probes according to hybridization characteristics of the candidate probes, forming at least one SuperCluster from the clusters; and selecting at least one probe from each SuperCluster for the probe array.

### BACKGROUND OF THE INVENTION

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection, differential gene expression analysis, identification of novel genes, gene mapping, finger printing, etc.) and proteomics.

In using such arrays, the surface-bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interrogated, for example, by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Generally, in discovering or designing probes to be used in an array, a nucleic acid sequence is selected based on the particular gene of interest, where the nucleic acid sequence may be as great as about 60 or more nucleotides in length, or as small as about 25 nucleotides in length or less. From the nucleic acid sequence, probes are synthesized according to various nucleic acid sequence regions, i.e., subsequences of the nucleic acid sequence and are associated with a substrate to produce a nucleic acid array. As described above, a detectably labeled sample is contacted with the array, where targets in the sample bind to complementary probe sequences of the array.

As is apparent, a step in designing arrays is the selection of a specific probe or mixture of probes that may be used in the array and which increase the chances of binding with a specific target in a sample, while at the same time reducing the time and expense involved in probe discovery and design. In practice, designing an optimized array typically involves iterating the array design one or more times to replace probes that are found to be undesirable for detecting targets of interest, either due to poor signal quality and/or cross-hybridization with sequences other than the targets of interest. Such iterations are costly and time consuming.

For example, conventional probe design may be performed experimentally or computationally, where in many instances it is performed computationally. Accordingly, probe design usually involves taking subsequences of a nucleic acid and filtering them based on certain computationally determined values such as melting temperature, self structure, homology, etc., to attempt to predict which subsequences will generate probes that will provide good signal and/or will not cross-hybridize. The subsequences that remain after the filtering process are selected to generate probes to be used in nucleic acid arrays.

While attempts have been made to predict which probes will provide the best results in an array assay, such attempts are not completely satisfactory as probes selected using these methods are often still found to be undesirable for one or both of the above-described reasons. In other words, some probes will still fail or give false results as the computational techniques used to filter and select the probes are not precise predictors. Accordingly, as mentioned above, typically an array design must be iterated a number of times in order to filter out all the undesirable probes from the array. Furthermore, such attempts often characterize probes after they have been synthesized, that is after time and expense have already been invested.

There is continued interest in the development of new methods and devices for producing arrays of nucleic acid probes that provide strong signal and do not cross-hybridize with sequences other than targets of interest.

### SUMMARY OF THE INVENTION

The invention provides methods, systems and computer readable media for identifying and selecting nucleic acid probes for detecting a target with a nucleic acid probe array or microarray. Embodiments comprise, in general terms: selecting a plurality of candidate probes; forming a plurality of clusters from the plurality of candidate probes according to hybridization characteristics of the candidate probes to a target sequence; forming at least one SuperCluster from the clusters; and selecting at least one probe from each SuperCluster for the probe array.

Methods, systems and computer readable media are provided for identifying and selecting nucleic acid probes for detecting a target with a probe array. Embodiments include: selecting a plurality of candidate probes from a consensus region associated with a plurality of nucleic acid transcripts; hybridizing nucleic acids from each of a plurality of tissue samples to each of a plurality of microarrays, each of said microarrays comprising said plurality of candidate probes; forming a plurality of clusters from said plurality of probes according to hybridization characteristics of said candidate probes across said different tissue samples; forming at least one SuperCluster from said clusters; and selecting at least one probe from each said SuperCluster for said probe array.

These and other advantages and features of the invention will become apparent to those persons skilled in the art upon reading the detailed description below.

### BRIEF DESCRIPTION OF THE DRAWINGS

FIG. 1 is a flow chart of a process of probe selection utilizing probe SuperClustering in accordance with the invention.

FIG. 2 is a diagram illustrating the relationship between GeneBin transcript sequences, consensus sequences and corresponding candidate probes in accordance with the invention.

FIGS. 3A-C show experimental Cluster results from the hybridization of candidate probes to multiple tissue samples in accordance with the invention.

FIG. 3D shows the hybridization results of candidate probes to tissue samples in which no cluster is formed.

FIG. 4 shows a graph illustrating the analysis of Clusters within a GeneBin in which two SuperClusters are identified in accordance with the invention.

FIG. 5 is a flow chart of a process of SuperClustering in accordance with the invention.

FIG. 6 is a block diagram illustrating an example of a generic computer system which may be used in implementing the present invention.

### DETAILED DESCRIPTION OF THE INVENTION

Before the present methods and systems are described, it is to be understood that this invention is not limited to particular genes, genomes, methods, method steps, statistical methods, hardware or software described, as such may, of course, vary. It is also to be understood that the terminology used herein is for the purpose of describing particular embodiments only, and is not intended to be limiting, since the scope of the present invention will be limited only by the appended claims. The invention is described primarily in terms of use with whole genome microarrays, and in particular the human genome. It should be understood, however, that the invention may be used with any group of transcripts that share some sequence identity.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a sample” includes a plurality of such samples and reference to “the microarray” includes reference to one or more microarrays and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source.

An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups).

A nucleotide “Probe” means a nucleotide which hybridizes in a specific manner to a nucleotide target sequence (e.g. a consensus region or of an expressed transcript of a gene of interest).

A “GeneBin” means a group of transcripts that share some sequence identity and overlap on the genome. One “GeneBin” may include more than one “gene”.

A “Consensus Region” means a sequence common to all or many transcripts within a “GeneBin”. Multiple “Consensus Regions” may be formed for one “GeneBin”.

A “Cluster” means a group of probes, designed from a single Consensus Region, that exhibit similar behavior across a range of gene expression experiments, as determined by a Pearson Correlation Coefficient algorithm or other algorithm for determining similarity.

A “SuperCluster” means a group of clusters of probes, designed from a single “GeneBin”, that exhibit similar behavior across a range of gene expression (experiments, as determined by a similarity algorithm.

An “array” or “microarray”, unless a contrary intention appears, includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one that is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location.

“Hybridizing” and “binding”, with respect to polynucleotides, are used interchangeably.

A “pulse jet” is a device which can dispense drops in the formation of an array. Pulse jets operate by delivering a pulse of pressure to liquid adjacent an outlet or orifice such that a drop will be dispensed therefrom (for example, by a piezoelectric or thermoelectric element positioned in a same chamber as the orifice). An array may be blocked into subarrays which may be hybridized as separate units or hybridized together as one array.

Any given substrate may carry one, two, four, or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features), each feature typically being of a homogeneous composition within the feature. Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of, for example, less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either polynucleotide precursor units (such as monomers) in the case of in situ fabrication, or the previously obtained polynucleotide. Such methods are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample (for example, a fluorescently labeled polynucleotide or protein containing sample), and the array is then read. Reading of the array may be accomplished by illuminating the array and reading the location and intensity of resulting fluorescence at multiple regions on each feature of the array. For example, a scanner may be used for this purpose that is similar to the AGILENT MICROARRAY SCANNER manufactured by Agilent Technologies, Palo Alto, Calif. Other suitable apparatus and methods are described in U.S. patent applications: Ser. No. 10/087,447 “Reading Dry Chemical Arrays Through The Substrate” by Corson et al.; and in U.S. Pat. Nos. 6,518,556; 6,486,457; 6,406,849; 6,371,370; 6,355,921; 6,320,196; 6,251,685; and 6,222,664. The above patents and patent applications are incorporated herein by reference. Arrays may also be read by other methods or apparatus than the foregoing, with other reading methods, including other optical techniques (for example, detecting chemiluminescent or electroluminescent labels) or electrical techniques (where each feature is provided with an electrode to detect hybridization at that feature in a manner disclosed in U.S. Pat. No. 6,251,685, U.S. Pat. No. 6,221,583 and elsewhere). A result obtained from the reading may be used in accordance with the techniques of the present invention in screening and finding multiple drug treatment therapies. A result of the reading (whether further processed or not) may be forwarded (such as by communication) to a remote location if desired, and received there for further use (such as further processing).

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

An important aspect of the invention is to differentiate “unique” from “redundant” sequences, i.e., to determine which transcript sequences belong to the same “gene.” Gene boundaries, and even the meaning of “gene”, are often ill defined. The term “GeneBin” is used by the inventors to describe a grouping of transcript sequences that show overlap of both the sequences themselves and overlap when mapped to a genome such as the human genome. A GeneBin will thus generally represent a single gene. However, in some cases, more than one gene will fall into a single GeneBin, while in other cases a single gene may be split into more than one GeneBin, depending on the population of transcripts that are included in the assembly. Consensus regions are determined from the GeneBins, and are utilized for selection of candidate probes for a microarray. These candidate probes are formed into clusters based on hybdridization characteristics of the probes, and the clusters are formed into SuperClusters in accordance with the invention. Use of SuperClusters as provided by the invention eliminates probe redundancies and provides for optimal use of the available “real estate” on microarray substrate surfaces as described more fully below.

The present invention provides alternative and novel methods and systems for probe microarray selection that overcome the drawbacks of existing microarray probe selection techniques. The methods of the instant invention utilize probe/target hybridization experiments and unique data analysis techniques to identify and select nucleotide probe(s) that target transcripts actually expressed in various tissues or samples of interest. The methods for probe selection described within, will benefit from flexible microarray fabrication technologies that can rapidly customize array content, as more information is forthcoming on the human genome.

The invention provides methods, systems and computer readable media for identifying and selecting nucleic acid probes for detecting a target with a nucleic acid probe array or microarray. The methods comprise, in general terms: selecting a plurality of candidate probes; forming a plurality of clusters from the plurality of candidate probes according to hybridization characteristics of the candidate probes to a target sequence; forming at least one SuperCluster from the clusters; and selecting at least one probe from each SuperCluster for the probe array. The hybridization characteristics for the plurality of probes may be measured using a plurality of different tissue samples comprising the target sequence.

Identification of outlier probes not associated with any of the clusters may be performed. Further, identification of outlier probes each associated with one of the clusters may be performed, based on a metric different from a metric used for forming the clusters. An example of such a different metric that may be used is Euclidean distance measurement. Identification of SuperCluster outliers not associated with the SuperCluster may also be performed.

A plurality of nucleic acid transcripts may be compiled and aligned to identify any sequence redundancy in the transcripts, and to identify a consensus region for the plurality of transcripts. The plurality of candidate probes are associated with the consensus region.

The invention is particularly useful with whole genome microarrays, such as microarrays based on the human genome. The invention permits more cost-effective and efficient identification of gene expression patterns which can be associated with human disease, points of therapeutic intervention, and potential toxic side-effects of proposed therapeutic entities. Prior to the introduction of the whole genome microarray, most commercial microarray offerings (and home-spotted microarrays as well) required at least two microarrays to provide coverage of most of the human genome. An entire genome may be encompassed by a single microarray at significant cost saving, as processing of the single microarray requires smaller amounts of costly reagents and reduced time using detection instruments and associated software.

The present systems, techniques, methods and computer readable media also provide for streamlined workflow, since researchers need only to prepare and process one microarray instead of two or more per sample, with fewer steps in processing and tracking required.

Further, greater reproducibility of results is provided for, since all data for an entire genome is generated from a single microarray, resulting in less variability in the data. When two or more microarrays associated with the same sample are processed separately, there are always questions of variability of the experimental conditions used to process each microarray.

Still further, smaller sample amounts are required when practicing the present methods, which is advantageous in cases (such as biopsies) where sample quantity is limited.

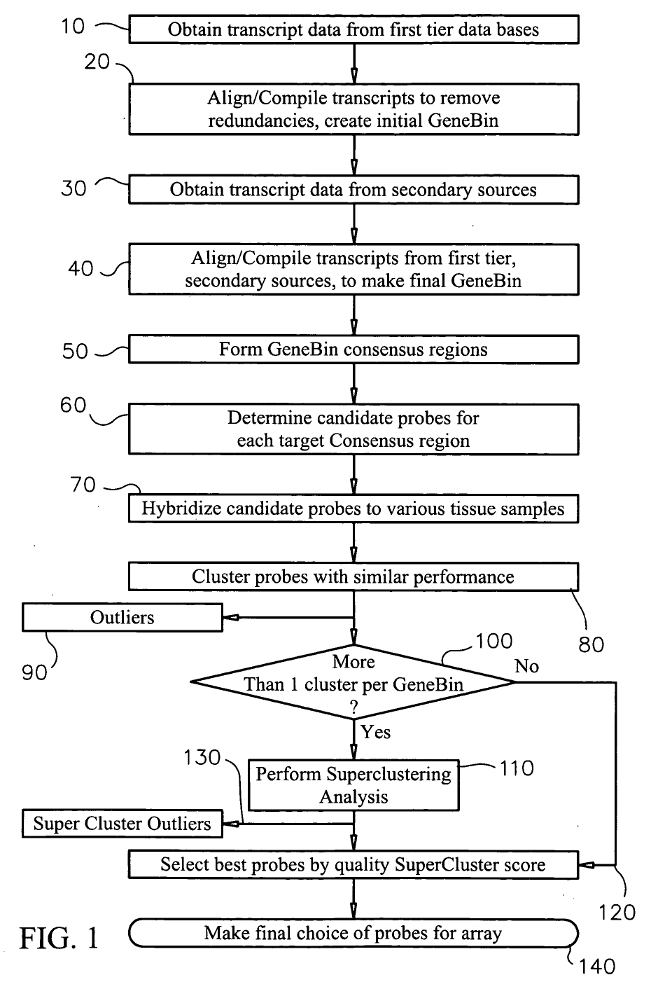

Referring now to FIG. 1, there is shown a flow chart of events that may be carried out in a nucleic acid probe selection method in accordance with the invention. At event 10, a first generation or first tier of nucleic acid transcripts is obtained from reliable database sources. The first phase event 10 focuses on data sources or databases that consist primarily of high-quality, full-length transcript sequences. These data sources include, but are not limited to, Incyte Foundation Full Length (FL) and Alternative Full Length (AltFL) databases, RefSeq, Ensemble cDNAs, and GenBank mRNA sequences that map to known proteins.

At event 20, the transcripts obtained in event 10 are aligned and compiled to assemble first or initial GeneBins. Aligning and compiling transcripts in event 20 identifies any redundant sequences associated with the transcripts of event 10. Sequence comparisons for this initial GeneBin assembly phase may be performed, for example, using BLAT (BLAST Like Alignment Tool, Genome Res. 12:656-664) available from Kent Informatics. The sequence data in the multiple data sources are compared to themselves (e.g., all of RefSeq to all of RefSeq), to the sequence data from all the other first tier sources of event 10, and to the genome of interest (for example, the human genome version NCBI33, April 2003). Representative transcript-to-genome alignments may be selected using “pslReps”, a program available from University of California, Santa Cruz, that detects best alignments in the genome with respect to local percent identity and discards alignments that are not within a certain percentage of the local best. Details of the BLAT parameters, tools, and post-processing can be found at http://www.cse.ucsc.edu/˜kent/exe/usage.txt.

Identical sequences are expected to be found in event 20, as the sources of some of the data in event 10 are redundant. For example, Incyte Foundation encompasses most of RefSeq in the FL and AltFL databases, and thus many RefSeq sequences are identical to FL or AltFL transcripts. After the removal of identical sequences in event 20, transcripts are grouped into GeneBins. GeneBin assembly may proceed in the order of data sources listed above, with the initial GeneBins defined by the Incyte Foundation gene structure, with redundant Incyte genes collapsed together following BLAT comparisons of the FL and AltFL sources.

In general, sequences are determined to be redundant (i.e., fall into the same GeneBin) if they meet the criteria for a quality of ‘hit’ for a transcript-to-transcript BLAT and if the sequences map to overlapping regions of the genome. A transcript will map into a GeneBin if it meets the criteria for transcript-to-transcript and genome overlap with any transcript previously mapped to that particular GeneBin. For each data source, the sequences are compared to those in the previous data sources, and the new transcripts are clustered into the existing GeneBins, or used to start new GeneBins, as appropriate.

Following the initial round or phase of GeneBin assembly in event 20, some transcripts may map to multiple GeneBins. This outcome may occur when there is a “bridging” transcript present in a later data source that “bridges” the transcripts present in the earlier sources. For this reason, following the initial GeneBin assembly the GeneBins go through a process of “GeneBin collapse” where GeneBins are combined on the basis of sharing a transcript. The “GeneBin collapse” process may create GeneBins containing more than one gene, a deficiency which can be resolved later on during the probe selection process. These “collapsed GeneBins” may be further analyzed for consensus sequence generation as described further below.

To include additional transcripts without contaminating results from high quality, first tier sequences of event 10, a second round of GeneBin creation is performed in event 30 using databases of sequences which are not as well annotated and which may contain non-full length transcript sequences. In event 30, transcript data is obtained from secondary sources, such as proprietary or customer databases or “partials” databases. This second phase of GeneBin assembly utilizes sequences acquired from lower confidence data sources that might contain partial transcript sequences. Such secondary data sources include, for example, the Incyte Foundation partials database (from which only partial transcripts are mapped into GeneBins), Genbank mRNAs that do not map to know proteins, Unigene Representative sequences not included as first tier sequences, and may also include customer requested sequences.

In event 40, the second tier transcript sequence data of event 30 is mapped to the first phase GeneBins generated in event 20, and formed into new GeneBins using the same process and criteria outlined above in event 20. Following the initial mapping into GeneBins, transcripts from these second tier data sources that map to round one GeneBins (i.e. redundant transcripts) are discarded.

An additional round of “GeneBin collapse” is carried out on the second phase GeneBins of event 40 to combine GeneBins sharing a transcript. A smaller subset of GeneBins are selected for consensus region generation and probe design as described below. The GeneBins are selected based on the transcripts within the GeneBins mapping to the genome with multiple exons, or public annotation of the transcripts in repositories such as TIGR Resourcerer (http://pga.tigr.org/tigr_scripts/magic/rl.pl) that indicate that customers may be interested in probes to those transcripts. Consensus regions are typically suitable for input into Probe Design (e.g., must be within, for example, 1200 bases of 3-prime end).

In event 50, consensus regions are generated from the second phase GeneBins of event 40. Each consensus region generated in event 50 represents many or all of the transcripts in the corresponding GeneBin of event 40. Multiple consensus regions may be generated from a single GeneBin when necessary to represent all transcripts within a GeneBin. A consensus region is sequence pattern derived from the alignment of the multiple transcripts in a Genebin that represents the common nucleotides among the transcripts at each position in a sequence. Gaps and mismatches in alignments are represented as “N”s.

Consensus regions are generated using the assumption that most of the transcripts within a GeneBin will be overlapping. However, not all transcripts may be found to be sufficiently overlapping to generate a single consensus region large enough for design of sufficiently independent probes for use in a microarray. Also, probes designed to represent a given transcript need to be sufficiently close to the 3′ end of the transcript to detect that transcript given the 3′ end bias of many target labeling techniques. This 3′end distance requirement needs to be accounted for in the consensus region generation process. Further, some GeneBins may contain transcripts that do not overlap at all, but which are binned together on the basis of a bridging transcript. For these reasons, the consensus region generation in event 50 is flexible enough to allow for multiple consensus regions to be designed for a single GeneBin.

In event 60, candidate probes are selected. The purpose of the probe design or selection process is to choose probe sequences that will uniquely and sensitively probe for the particular transcripts that fall within a GeneBin in any sample of interest. Over the last several years, rather standard processes have evolved for the theoretical identification of good probe sequences for use on DNA arrays (see, for example, Bozdech Z, et al. Genome Biology 4, R9; Hughes T R, et al. Nature Biotechnology 19, 342-7, 2001; and Nielsen H B, et al. Nucleic Acids Research 31, 3491-3496, 2003). Probes must be chosen that will all hybridize sensitively and specifically under common hybridization conditions shared by all oligonucleotides on a particular microarray. This requirement is very demanding, as there are tens of thousands of probes on an array which must work well under the same conditions. In many embodiments the present invention utilizes probes which are between 40 and 80 nucleotides in length, and in certain embodiments, probes which are oligonucleotides 60 nucleotides in length may be used. The longer probe length enables greatly enhanced sensitivity relative to shorter oligonucleotides while still maintaining adequate selectivity so as to be able to distinguish different gene transcripts.

To further the selection of candidate probes in event 60, the target sequences comprising the consensus regions of the GeneBins are purged of any repetitive sequences that are known to exist throughout the whole genome of interest which may compromise probe specificity. A screening process may be used which looks for particular putative probe sequences within a consensus region that possess a desirable base composition. The probes are then checked for specificity by comparison of the candidate probe sequences against the similarity database using a thermodynamic model seeded by BLAST. Any probe with ‘hits’ (other than against the transcripts of the GeneBin from which the probe originates) is discarded, as a ‘hit’ would imply cross hybridization and thus a lack of specificity.

Several candidate probes for each consensus region may be chosen in event 60, and then the list of candidate probes may be refined through empirical measurements. For consensus regions generated from second tier databases of event 30, additional criteria (e.g., competitive content, mapping to genome with multiple exons) may be used, to limit the number of test arrays required. The empirical measurements employed by the invention in event 60 typically involve a process which rewards self-consistent behavior with respect to measuring gene expression across ten or more separately chosen candidate probes within a given target consensus sequence.

In event 70, the candidate consensus region probes of event 60 are hybridized to multiple tissue/cell line combinations, such as brain versus lung, lung versus kidney, kidney versus HeLa, etc., to determine which candidate probes detect expression products of the consensus region in a reliable fashion across the various tissues/cell lines.

In the clustering of event 80, probe hybridization data is subjected to CAST clustering algorithms (Ben-Dor A., et al (1999), J. Comput. Biol. 6, 281-297), to identify probe consensus regions that cluster with regard to Pearson Correlation of Log Ratio values across experiments. Alternative correlation measures, such as Kendall's rank correlation can also be utilized. Each cluster formed in event 80 represents a group of probes, designed from a single consensus region that exhibits similar behavior across the range of gene expression experiments of event 70. Further description of cluster processing techniques that may be employed are include in co-pending, commonly assigned application Ser. No. 10/303,160 filed Nov. 22, 2002 and titled “Methods for Identifying Suitable Nucleic Acid Probe Sequences for Use in Nucleic Acid Arrays”. Application Ser. No. 10/303,160 is hereby incorporated herein, in its entirety, by reference thereto.

Following clustering based on Pearson correlation, probes within clusters may be marked as outliers based on the Euclidean distance, as described in more detail below.

In event 100, a determination is made as to whether or not more than one cluster exists for each GeneBin. If a GeneBin is found to have more than one cluster, SuperCluster analysis is carried out in event 110. If a GeneBin is determined to have only one cluster of probes, no further cluster analysis is needed and further probe selection is carried out in event 120.

The query of event 100 determines if two or more different expression behaviors are exhibited within a given GeneBin. Where two or more clusters from event 80 show similar expression behavior, through combining and differentiating the probe clusters, and thus consensus regions, based on the probe clusters expression profiles, those clusters are formed into a SuperCluster in event 110. The presence of different probe behaviors within a GeneBin, as identified by a failure to form one single SuperCluster, might suggest for example, alternative splicing (including alternative 3′ exons), alternative poly adenlylation sites, or overcollapsed GeneBins (i.e., non-optimal transcript data compilation in event 20 and/or event 40). In any instance where two or more different probe cluster behaviors are found, multiple probes for that GeneBin can then be included on the final array design.

In the SuperCluster formation of event 110, it is possible that one or more probe clusters do not fit within the profile of the identified SuperClusters. Such “SuperCluster outlier” probes may be identified in event 130 and excluded from probe selection in event 120, or used in subsequent SuperCluster analysis.

Once the probes have been identified with a specific SuperCluster, further probe selection for the microarray is carried out in event 120. The probe selection of event 120 may be based on cluster quality, SuperCluster quality, and probe target information.

In event 140, final probe selection for a whole microarray is carried out. Final probe selection for a whole genome microarray may comprise, for example, the selection of probes to represent all SuperClusters, additional probes as necessary to ensure coverage of data sources deemed representative of the “whole genome”, defining and compiling appropriate descriptive information about the selected probes for annotation of the microarray. The probe validation and optimization strategy for probe selection for a microarray as shown in FIG. 1 and described above is based on the assumption that probes for a specific target should show similar differential expression behavior within a single experimental set and across multiple experimental sets. A more detailed description of various events depicted in FIG. 1 is given below.

Candidate Probe Selection

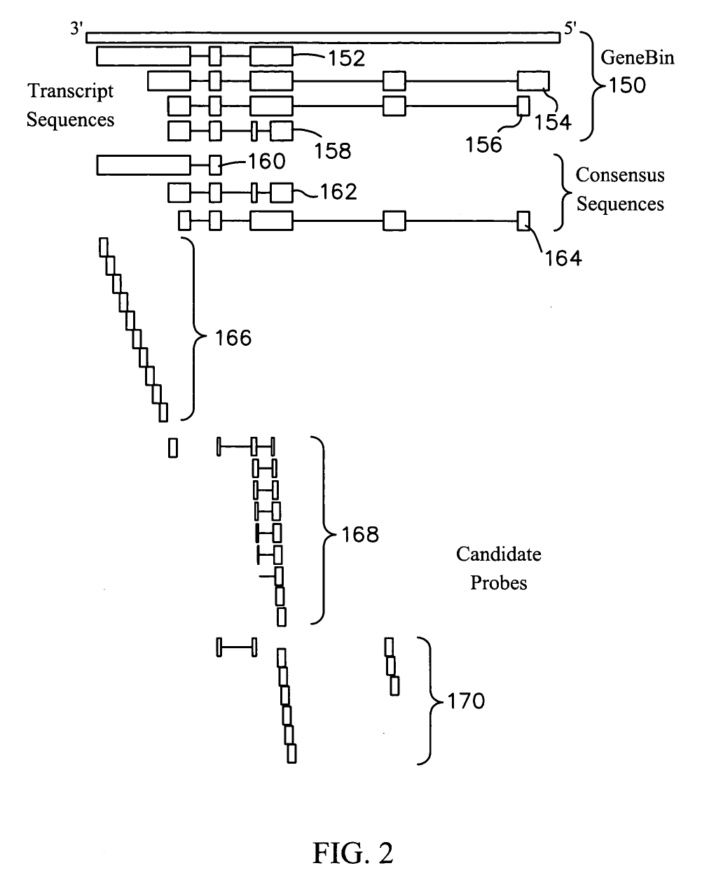

FIG. 2 is an illustration showing the relationship between GeneBin transcripts identified in event 40, consensus sequences determined in event 50, and the selection of candidate probes for each target consensus region in event 60 of FIG. 1. FIG. 2 shows a GeneBin 150 with four transcript sequences 152, 154, 156, and 158 which are identified by the alignment and compiling methods described above for event 40 of FIG. 1. Transcripts 152, 154, 156, and 158 are further analyzed in event 50 of FIG. 1 to generate consensus sequences 160, 162 and 164 which cover all of the four transcript sequences 152, 154, 156, and 158 of GeneBin 150.

The consensus sequences or regions 160, 162 and 164, are analyzed to identify sequences appropriate of candidate probes which would cover all of the consensus regions 160, 162 and 164 of GeneBin 150. Three groups of candidate probes 166, 168 and 170 are selected in the example provided by FIG. 2, with one probe group for each of consensus regions 160, 162 and 164, respectively. Candidate probes 160, 162, 164 are selected by the methods and processes described above for event 60 of FIG. 1. Each group of candidate probes 166, 168 and 170 shown in FIG. 2 comprise ten candidate probes all related to a specific consensus sequence. Probes in group 166 are generated for consensus region 160, while probes in group 168 are generated for consensus region 162, etc.

Hybridization of Candidate Probes

Once the candidate probes for a GeneBin have been selected, the candidate probes are hybridized to a plurality of tissue/cell line samples as depicted by event 70 in FIG. 1. During the hybridization experiments, candidate probes are placed on a test microarray and subjected to target sequences derived from various tissues or cell lines, as described above. The candidate probes are experimentally tested for their ability to detect the target consensus sequences from which they were derived. Each candidate probe/tissue pair may be tested multiple times through hybridization experiments to determine which probes show consistent differential expression over numerous experiments. One example of a test microarray is the hybridization of ten candidate probes to ten different tissue/cell line combinations (with at least four replicates per sample pair): one self vs. self sample pair and nine additional non-self vs. self tissue sample pairs. A self vs. self pair is a tissue pair in which the same tissue is used for both halves (e.g., spleen vs. spleen or lung vs. lung, etc.) A non-self vs. self pair is a tissue pair in which different tissues are used for the halves (e.g., heart vs. spleen or lung vs. spleen, etc.). “Good” probes will show mutually consistent differential expression across the different tissue samples tested.

The arrays, after hybridization, are scanned and the feature data is extracted using Agilent Technologies Feature Extraction software (Agilent Technologies, Inc., Palo Alto, Calif.) or like programming.

Probe Clustering

The clustering analysis is the process that detects mutually consistent differential expression across the different tissue sample pairs tested, or lack thereof. Thus, all the data is subject to cluster analysis to determine the “good probes”. The strategy of using clustering analysis for experimental probe validation is based on the assumption that probes that hybridize to a single target will behave similarly in gene expression experiments, both within a single experimental probe/tissue pair and across multiple experimental pairs. Disparate Log ratio values for probes designed to a single target may be caused by a variety of factors that include non-specific hybridization of additional target(s), probe secondary structure or other factors that limit hybridization efficiency, mis-annotation of target structure (e.g., intron/exon boundaries), unrecognized alternative splicing and labeling biases. Most, if not all, of these factors cannot be accurately predicted solely by consideration of the transcript sequence.

Various clustering techniques may be used in the analysis of gene expression data to identify genes that are co-regulated. For probe validation, CAST (Cluster Affinity Search Technique) clustering algorithms (Ben-Dor A., et al (1999), J. Comput. Biol. 6, 281-297) may be used to identify co-regulated probes from the candidate probes designed to target a single consensus region. CAST is a non-greedy clustering algorithm that constructs clusters by preserving a high intra-cluster similarity at all stages. This level of similarity is determined by an input parameter τ. The CAST algorithms have several advantages over other clustering algorithms useful for this application. Most importantly, the CAST algorithms form a non-hierarchical clustering (i.e.: the clusters are unrelated and cluster boundaries are determined by the algorithm). Also, these algorithms do not assume a given number of clusters (i.e.: the number of clusters is determined by the algorithm instead of being a constant number given as an input parameter).

When analyzing probes for targets for a whole genome, numerous rounds of the CAST clustering application may be needed. During the first rounds of clustering, the CAST threshold and score criteria are gradually altered as the rounds proceed, and clusters formed in these first rounds are considered “high confidence clusters”. During the later CAST clustering rounds, the CAST threshold values and score criteria are gradually altered, as well as the reduced “probe span” requirement compared to the first rounds of clustering. Most if not all of the clusters produced in these later rounds tend to be of lower quality, and are considered “moderate confidence clusters.”

Specific events that are carried out in the cluster analysis of event 80 generally include prefiltering of data for dye-biased probes, generation of an expression matrices, calculation of a “Similarity Matrix”, as well as the actual clustering of probes using CAST. These specific operations are described below.

Prefiltering of Data for Dye-Biased probes

Prior to the initiation of cluster analysis, probes may be “ear-marked” or otherwise identified if they exhibited dye-bias in self vs. self hybridization experiments, using both log ratio and P-value criteria. Probes that are marked as dye-biased are not selected as final probes.

Generation of Expression Matrix

To generate an expression matrix, replicate log ratio values for a given sample probe/tissue pair are combined using error-weighted averaging. The combined log ratio data for candidate probes designed to target a single gene are used to populate an expression matrix I, where Iᵢⱼ is the measured expression level of probe i in experiment (condition) j. Only those probes that exceed a user-specified signal threshold on at least one of the combined test arrays are included in the expression matrix. The size of the expression matrix required for robust analysis is dependent on the similarity measure used in the clustering algorithm. For example, the significance of the Pearson's correlation coefficient depends on the number of experiments, and an expression matrix consisting of at least 8 experiments is preferred. Performance of the clustering algorithm does not depend on the number of probes, since probes are assigned to clusters based on the affinity to cluster. However, the number of probes should be high enough to be representative of all possible probes for the input (target) sequence.

Calculation of Similarity Matrix S

In a similarity matrix, the entry Sᵢⱼ represents the similarity of the expression pattern for probes i and j. The similarity measure used by CAST for this operation is independent of the clustering mechanism. The Pearson's correlation coefficient and the Kendall's rank correlation are examples of similarity measures useful in the present invention.

Clustering of Probes Using Cast

The CAST clustering algorithms partition the candidate probes into groups based on similar expression patterns. The input into the algorithm is a pair (S,τ) where S is an n-by-n similarity matrix and τ is a user-specified affinity threshold that determines what affinity level is considered significant. The algorithm constructs clusters incrementally and uses average similarity (affinity) between unassigned vertices and the current cluster to make its next decision to add or remove elements from groups. The clusters are “stable” when the average similarity exceeds the affinity threshold (τ). In practice, the cluster analysis is performed at decreasing affinity thresholds until a cluster meeting user-defined criteria (such as minimum size, probe span, and score) is formed. Cluster membership is assigned for each cluster and a cluster size and a cluster quality score is calculated. The quality score of a cluster is a measure of the likelihood of such a cluster occurring if data from unrelated probes from the data set were clustered. High probability clusters (i.e.: those where the data clusters much more tightly grouped than would be expected from randomly selected data) are given high scores. If there is only one cluster found within a GeneBin, then no further analysis is carried out and a probe(s) is selected by quality of cluster score and other criteria as shown in event 100 and 120 of FIG. 1. If more than one cluster is found within the GeneBin, then further analysis of the clusters is completed to identify SuperClusters as depicted by event 110 of FIG. 1.

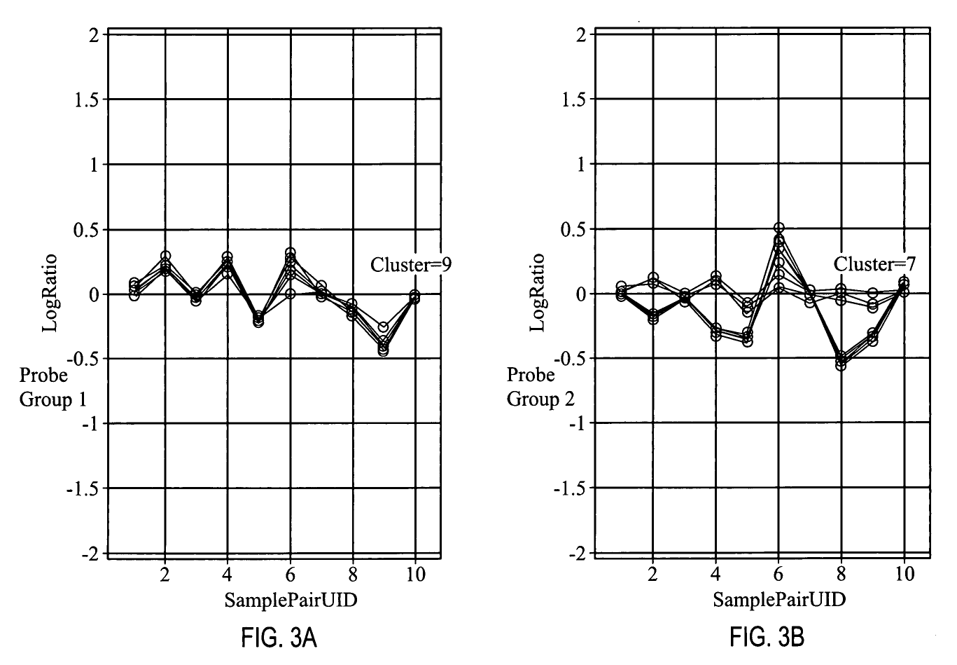

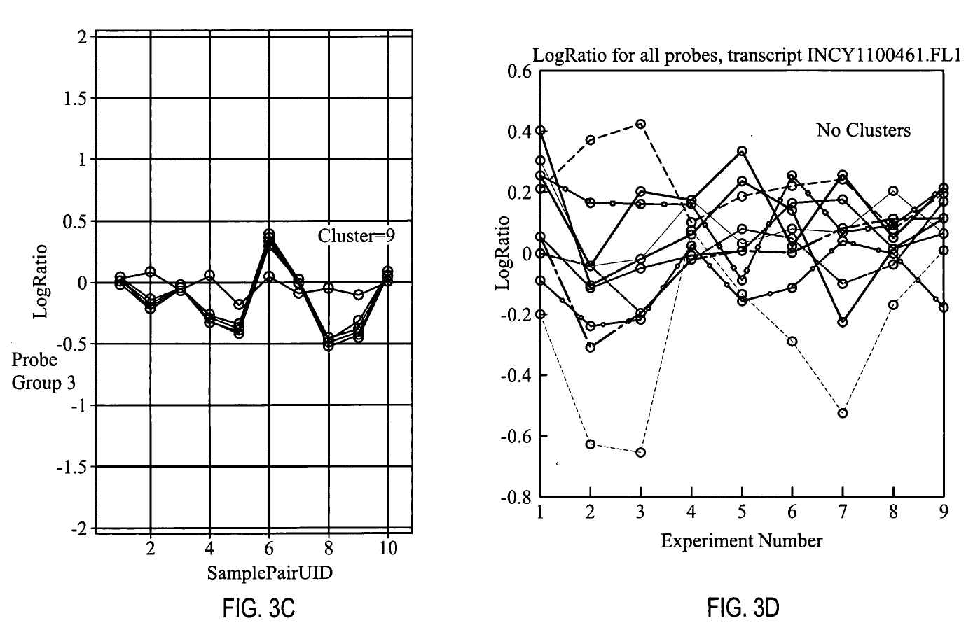

Possible clustering outcomes of data generated from hybridization experiments of event 70 are shown in FIGS. 3A-3D. FIGS. 3A-C show expression graphs representative of the three groups of candidate probes 166, 168 and 170 shown in FIG. 2, respectively. FIG. 3A, shows an expression graph where nine of the ten candidate probes in probe group 166 have similar expression ratio results, indicating that these probes respond almost identically across each of the plurality of tissue samples (tissue pairs). Since the probes are independent of each other, cross hybridization from non-specific mRNA sequences would show up as aberrant behavior across the different tissue pairs (based on the assumption that different levels of different mRNAs are present in each tissue). The expression data shown in FIG. 3A, identifies one cluster with nine candidate probes of probe group 166 as cluster members.

FIG. 3B is a graph showing possible hybridization results representative of the clustering of candidate probes within probe group 168 of FIG. 2. FIG. 3B shows a situation in which there is one cluster of probes with seven probe members. The other three probes show similar expression patterns with each other, but do not meet the criteria necessary to form a cluster.

FIG. 3C is a graph showing expression results, which are representative of a possible hybridization result of probe group 170 shown in FIG. 2. Here all of the candidate probes in probe group 170 form a single cluster, except for one candidate probe. The one probe excluded from the cluster shown in FIG. 3C may be detecting a unique sequence within the GeneBin or it may be performing differently due to cross-hybridization or for other reasons.

Probes are considered to be outliers based on Euclidean distance measurements first in Log Ratio space, and then in signal intensity space. Euclidian distance measurements in Log Ratio space are weighted by the RMS value of the log ratio to more likely identify probes with compressed Log Ratios as outliers. The Euclidean distance is measured for the entire cluster, and for the cluster missing each candidate probe. A probe is considered a Euclidean outlier only if the new distance (without the probe) is significantly smaller than the old distance. The process is then repeated with the new cluster, until no more probes can be removed, a minimum distance is obtained, or the number of probes in the cluster has reached a defined threshold. Following the Log Ratio outlier removal, a similar process may be repeated in signal intensity space. User defined parameters may include the threshold for Euclidean distance difference following removal of a given probe, the minimum cluster size following outlier removal, and the minimum Euclidean distance below which no outliers are called. The Euclidean distance metric for a cluster is defined as the average value of all probe-to-probe distances for probes in the cluster, where distance is calculated as the square root of the sum of squares of the differences in Log Ratio or Signal Intensity, and the average value is scaled by the root mean square Log Ratio or Signal Intensity of all probes in the cluster.

There are situations in which no candidate probes exhibit clustering or correlated behavior for consensus region of a GeneBin. FIG. 3D is an expression graph of ten candidate probes which do not form any clusters. This situation may arise when the underlying sequence information for a GeneBin is of marginal quality, when the gene or transcript of interest is expressed at a very low level in the selected tissue samples, or when the gene or transcript is not sufficiently differentially expressed in the selected tissue. The clusters determined by the above events, and represented in FIGS. 3A-C, are further analyzed for SuperClustering prior to final probe selection as depicted in event 110 of FIG. 1.

SuperClustering of Clusters

SuperClustering involves the assembly of new clusters using the old clusters within a GeneBin as a starting point. Any probe that is not included in a cluster during the original clustering round is not included in SuperClustering. However, Euclidean cluster outliers may be included in SuperClustering rounds.

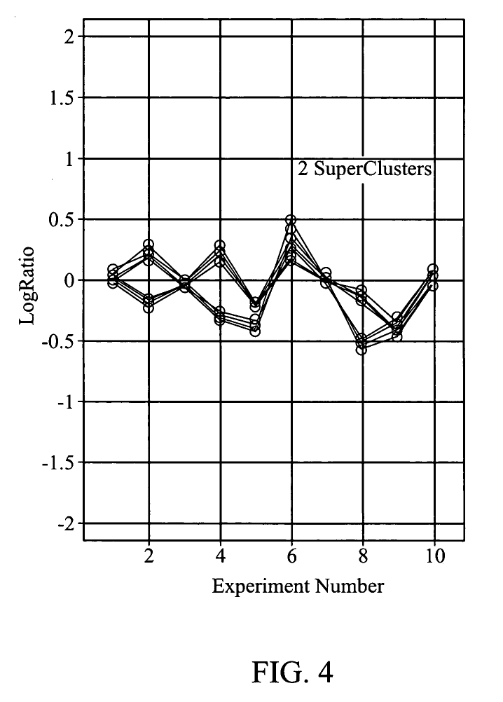

This second level of clustering, or “SuperClustering” is illustrated graphically in FIG. 4. SuperClustering allows for the differentiation between truly unique cluster behavior, which might indicate a real alternative transcript, and similar cluster behavior, which might suggest incomplete transcript sequences leading to the formation of redundant consensus regions or targets. FIG. 4 is an expression graph which exemplifies the SuperClustering of the clusters and possible outliers identified in FIGS. 3A-C. Two SuperClusters are identified and shown in FIG. 4. As described below, the probe clusters of a GeneBin are analyzed for possible SuperClustering with the other probe clusters within a GeneBin. When testing groups of candidate probes within a GeneBin or a whole genome for SuperClustering, all clusters to a GeneBin must be tested.

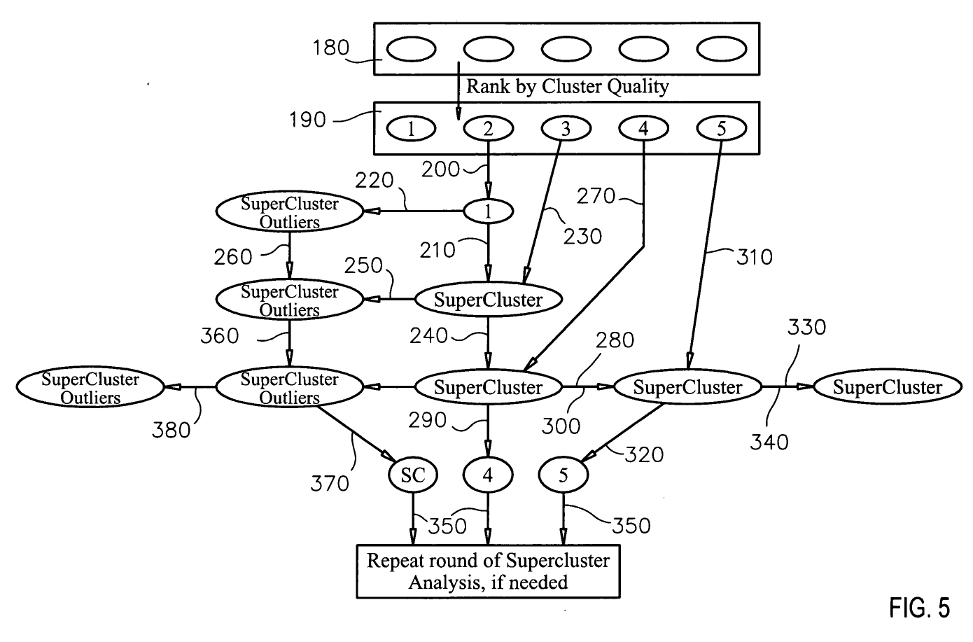

Referring now to FIG. 5, a diagram illustrating a method of SuperClustering is shown schematically in accordance with the present invention. At event 180, clusters within a GeneBin are obtained using the methods described for cluster formation above. At event 190, the clusters within the GeneBin are ranked by quality based on the CAST round in which the cluster was identified. Clusters may also be ranked in event 190 by cluster score information. The quality score of a cluster is a measure of the likelihood of such a cluster occurring if data from unrelated probes from the data set were clustered. High probability clusters are clusters where the cluster data of probes is much more tightly associated than would be expected from randomly selected data. High probability clusters are given a high score.

Five clusters shown at event 190, are ranked 1 through 5, with cluster 1 being the highest ranked cluster in the GeneBin and cluster 5 being the lowest ranked cluster. At event 200, candidate probes from cluster 1 and cluster 2 are re-clustered using an algorithm such as CAST, with a cluster threshold set below thresholds used originally to form the clusters, to form a SuperCluster at event 210. The threshold for SuperClustering may be about 10% below the lower of the two original cluster thresholds, but generally will not be less than about 70%. For the clusters 1 and 2 to continue as a SuperCluster in event 210, the SuperCluster must contain a sufficient proportion of each of the original clusters probes (e.g. 70% of the probes). If the new cluster formed by clusters 1 and 2 does meet these criteria, they form a SuperCluster in event 210. Probes from cluster 1 or 2 that do not fall into the new SuperCluster are labeled as SuperCluster outliers at step 220. If the clusters 1 and 2 do not meet the SuperCluster threshold, then cluster 2 is set aside (not shown in FIG. 5) until all other clusters (e.g. 3, 4 and 5) have been tested for SuperClustering with cluster 1. If cluster 2 does not form a SuperCluster with cluster 1, cluster 2 is used as the seed of a new round of SuperCluster analysis, and any subsequent clusters that have not joined the first SuperCluster produced with cluster 1, are tested for SuperClustering with this new SuperCluster (generated from cluster 2).

At event 230, the newly formed SuperCluster of event 210 is subjected to SuperCluster analysis for possible SuperCluster formation with cluster 3 from the GeneBin. Similar SuperCluster analysis is performed as described above, and the probes of cluster 3 are considered either in a SuperCluster (with clusters 1 and 2) at event 240, or as SuperCluster outliers at event 250, or the cluster does not join the SuperCluster and is set aside, as described above for cluster 2. Any SuperCluster outliers produced at event 220 and those produced at event 250 are set aside at event 260 for possible future SuperClustering analysis. SuperCluster outliers will only be used in further SuperClustering if a majority of the original cluster members are removed as SuperClusters during the rounds of SuperCluster analysis. If a majority of probes are removed as SuperCluster outliers, then all the probes for that cluster are removed from the SuperCluster, and the original cluster is returned to the pool of available clusters.

SuperClustering continues with another act of SuperClustering analysis at event 270, where cluster 4 of the GeneBin is analyzed with the SuperCluster formed from clusters 1-3 at event 240. Again, SuperClustering may generate a larger SuperCluster including probes from cluster 4 at event 280, while forming a larger SuperCluster (not shown in FIG. 5); or cluster 4 may not meet the threshold for SuperClustering and is set aside at event 290 for later rounds of SuperClustering analysis with the rest of the clusters that are not included in the SuperCluster seeded by cluster 1. In the later case, when cluster 4 does not meet the SuperCluster threshold, the SuperCluster formed from clusters 1-3 at event 240 stays untouched at event 300 for SuperCluster analysis with cluster 5.

At event 310, cluster 5 of the GeneBin and the SuperCluster identified at event 280 or 300 (depending on if cluster 4 was incorporated into the SuperCluster), are analyzed with another act of SuperCluster analysis. The schematic shown in FIG. 5, shows cluster 5 not meeting the threshold for SuperCluster formation and cluster 5 is left untouched at event 320 and the SuperCluster produced at event 280, 300 is left unchanged at event 330. It should be noted that if cluster 5 meets the SuperCluster threshold and combines with the SuperCluster at event 280 or 300, a larger SuperCluster would be formed at event 340 which included probes from cluster 5, and additional new SuperCluster outliers may also be identified (not shown in FIG. 5).

Another round of SuperCluster analysis, similar to that described above for cluster 1, is carried out on the clusters set aside, such as cluster 4, which did not form a SuperCluster at event 290. Cluster 4 at event 350 repeats the SuperClustering analysis with other clusters which did not form a SuperCluster with cluster 1. For example, if cluster 5 did not form a SuperCluster at event 320, cluster 4 and 5 would be subjected to SuperCluster analysis at event 350. If a cluster has still not formed a SuperCluster with another cluster from the GeneBin after subsequent rounds of SuperClustering, that cluster becomes a SuperCluster by itself. The cluster(s) determined to be associated with SuperClusters formed by the SuperClustering method, are further analyzed for probe validation and probe coverage for a specific GeneBin and gene product.

To briefly summarize SuperCluster analysis, multiple clusters are formed within a given GeneBin due to potential redundancy both from the consensus region generation and from the inclusion of previous probe data from already existing human genome microarrays. SuperClustering is a method and process of comparing clusters in a GeneBin to determine when two or more different expression behaviors are exhibited within a given GeneBin. Where two or more different probe cluster behaviors are found by SuperClustering analysis (e.g. multiple SuperClusters are produced within the GeneBin), multiple probes (e.g. one probe for each SuperCluster) for that GeneBin can be included on the final array design.

SuperClustering involves the assembly of new clusters using the old clusters by ranking of all clusters within a GeneBin based on the round in which the cluster was formed and also score information. Probes from the first two highest ranking clusters are reclustered using the CAST algorithm and for the result to continue as a SuperCluster, the newly formed cluster must contain a sufficient proportion of each of the old clusters probes. If the newly formed cluster does not meet the outlined criteria for a SuperCluster, the result is discarded, and the first or highest ranking cluster is subjected to another act of SuperClustering analysis with the next or third highest ranked cluster (if applicable). The other, discarded cluster (e.g. the second ranked cluster) is set aside, into the pool of available clusters for further rounds of SuperCluster analysis. In each round of SuperClustering, any probes from a cluster which is incorporated into a SuperCluster, that do not met the SuperCluster threshold are not included in s the new SuperCluster and are labeled as “SuperCluster Outliers.” Following the completion of SuperCluster analysis on all the clusters in a GeneBin (i.e., each of the GeneBin's clusters has been tested, in rank order, for membership in that SuperCluster), any “cluster,” having a majority of probe members lost as SuperCluster Outliers or that is a cluster that has been set aside for that particular SuperCluster, is returned to the pool of available clusters for further SuperCluster Analysis to determine if that cluster may form another SuperCluster with other available clusters. The SuperClustering process is continued on the pool of available clusters, until all clusters are in a SuperCluster. A SuperCluster may contain as few as one cluster or as many as all the clusters within a GeneBin.

Probe Selection From SuperClusters

Probes are selected from the SuperClusters based on cluster quality and probe target information: The final selected probes may be used for a “gene-based” microarray, in which probes are selected based on the gene level data, unless evidence of different expression profiles is detected during SuperClustering (i.e., more than one SuperCluster was obtained per GeneBin). During the initial rounds of probe selection, probes are selected from the experimentally validated clusters, with one probe being selected per SuperCluster. Selection of probes from the validated clusters may proceed as an iterative process, with multiple rounds of probe selection. For each round of probe selection, one or more criteria were changed from the previous round, and the process continues until one probe is selected for each SuperCluster.

The criteria used for selection of probes form a SuperCluster include, but are not limited to: the round of validation of CAST clustering the original cluster containing the probe of interest was obtained or validated (probes obtained in lower or earlier rounds of probe clustering are selected over those that cluster in later rounds); if the probe has been previously arrayed on an already existing genome microarray; the number of GeneBins to which the probe of interest targets (probes hitting fewer GeneBins are selected above probes hitting more GeneBins); and cluster score, (probes from higher scoring clusters are selected over those from lower scoring clusters). Additional criteria may be used for probe selection as will be recognized by those skilled in the art.

The criteria used to exclude probes from final probe selection for a microarray include, but are not limited to: whether or not the probe is a Cluster Outlier (all Euclidean cluster outlier probes may be excluded from the validated rounds of probe selection); whether or not the probe is a Supercluster Outlier (all SuperCluster outlier probes are typically excluded from the validated rounds of probe selection); or whether or not a probe is dye biased (all probes determined to be dye biased during the probe validation are typically excluded from probe selection).

Once a probe from a given SuperCluster has been selected, typically no other probes for that SuperCluster are permitted to be selected. The probe selection continues until a probe is selected for each SuperCluster in the GeneBin, and ultimately the whole genome for genome-based microarrays. If there was more than one probe with the same values for all of the selection parameters, the probe is selected based on the Probe ID (i.e., random, but repeatable).

Probes which cluster and SuperCluster well, may be further selected based on considerations such as the number of probes in a cluster, the tightness of the cluster, and the span of the probes across the length of the transcript sequence. In the final design for the microarray, only one probe is typically chosen to represent a particular GeneBin unless there is strong evidence for multiple independent transcripts that could be assayed (e.g., as in the case of multiple distinct “SuperClusters” for a given GeneBin). In the case of such strong evidence, additional probes may be selected as needed. The design for the whole genome microarray may then be reviewed and augmented with probes to ensure that probes for all high confidence GeneBins, most notably the first round GeneBins, are included. Finally, appropriate descriptive information (annotation) for each of the probes is compiled to be included with the microarray.

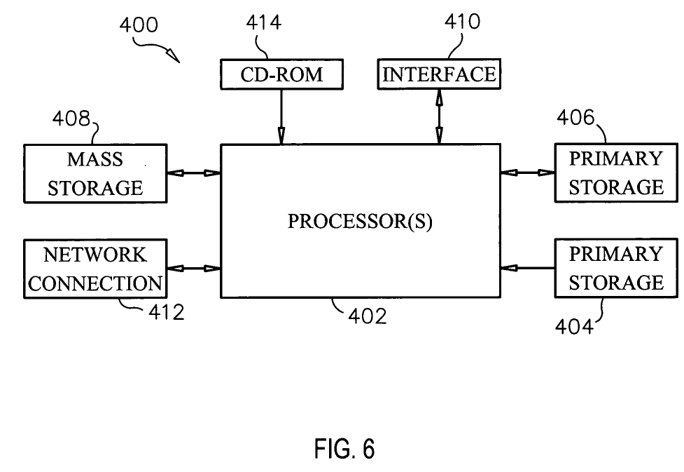

FIG. 6 illustrates a typical computer system 400 that may be used in processing events described herein. The computer system 400 includes any number of processors 402 (also referred to as central processing units, or CPUs) that are coupled to storage devices including primary storage 406 (typically a random access memory, or RAM), primary storage 404 (typically a read only memory, or ROM). As is well known in the art, primary storage 404 acts to transfer data and instructions uni-directionally to the CPU and primary storage 406 is used typically to transfer data and instructions in a bi-directional manner Both of these primary storage devices may include any suitable computer-readable media such as those described above. A mass storage device 408 is also coupled bi-directionally to CPU 402 and provides additional data storage capacity and may include any of the computer-readable media described above. Mass storage device 408 may be used to store programs, data and the like and is typically a secondary storage medium such as a hard disk that is slower than primary storage. It will be appreciated that the information retained within the mass storage device 408, may, in appropriate cases, be incorporated in standard fashion as part of primary storage 406 as virtual memory. A specific mass storage device such as a CD-ROM 414 may also pass data uni-directionally to the CPU.

CPU 402 is also coupled to an interface 410 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 402 optionally may be coupled to a computer or telecommunications network using a network connection as shown generally at 412. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for population of stencils may be stored on mass storage device 408 or 414 and executed on CPU 408 in conjunction with primary memory 406.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for identifying and selecting nucleic acid probes for detecting a target with a probe array, said method comprising:
selecting a plurality of candidate probes;
forming a plurality of clusters from said plurality of candidate probes according to hybridization characteristics of said candidate probes to a target sequence;
forming at least one SuperCluster from said clusters; and
selecting at least one probe from each said SuperCluster for said probe array.

2. The method of claim 1, wherein said hybridization characteristics for said plurality of probes are measured using a plurality of different tissue samples comprising said target sequence.

3. The method of claim 1, wherein only a single probe is selected from each said SuperCluster.

4. The method of claim 3, further comprising forming a microarray from said probes selected from said SuperClusters wherein said array includes only one probe from each said SuperCluster.

5. The method of claim 1, further comprising:
identifying clusters that do not belong to any SuperCluster; and
identifying at least one alternative splice form from said clusters that do not belong to any SuperCluster.

6. The method of claim 2, wherein said forming said plurality of clusters from said plurality of candidate probes comprises:
forming a plurality of microarrays, each said microarrays comprising said plurality of candidate probes;
hybridizing each of said plurality of microarrays to nucleic acids from each of said plurality of different tissue samples; and
clustering said candidate probes based on mutually consistent differential expression of said target sequence across said plurality of different tissue samples.

7. The method of claim 1, further comprising identifying outlier probes not associated with any of said clusters.

8. The method of claim 1, further comprising identifying outlier probes each associated with one of said clusters, based on a metric different from a metric used for said forming a plurality of clusters.

9. The method of claim 8, wherein said metric different from a metric used for said forming a plurality of clusters comprises Euclidean distance measurement.

10. The method of claim 1, further comprising:
compiling and aligning a plurality of nucleic acid transcripts to identify sequence redundancy in said transcripts; and
identifying a consensus region for said plurality of transcripts.

11. The method of claim 10, wherein said plurality of candidate probes are associated with said consensus region.

12. A method comprising forwarding a result obtained from the method of claim 1 to a remote location.

13. A method comprising transmitting data representing a result obtained from the method of claim 1 to a remote location.

14. A method comprising receiving a result obtained from a method of claim 1 from a remote location.

15. A method for identifying and selecting nucleic acid probes for detecting a target with a probe array, said method comprising:
selecting a plurality of candidate probes from a consensus region associated with a plurality of nucleic acid transcripts;
hybridizing nucleic acids from each of a plurality of tissue samples to each of a plurality of microarrays, each of said microarrays comprising said plurality of candidate probes;
forming a plurality of clusters from said plurality of probes according to hybridization characteristics of said candidate probes across said different tissue samples;
forming at least one SuperCluster from said clusters; and
selecting at least one probe from each said SuperCluster for said probe array.

16. The method of claim 15, further comprising identifying outlier probes not associated with any of said clusters.

17. The method of claim 15, further comprising identifying outlier probes each associated with one of said clusters, based on a metric different from a metric used for said forming a plurality of clusters.

18. The method of claim 17, wherein said metric different from a metric used for said forming a plurality of clusters comprises Euclidean distance measurement.

19. The method of claim 16, further comprising identifying SuperCluster outliers not associated with said SuperCluster.

20. The method of claim 15, further comprising:
compiling and aligning a plurality of nucleic acid transcripts to identify sequence redundancy in said transcripts; and
identifying a consensus region for said plurality of transcripts.

21. The method of claim 20, wherein said plurality of candidate probes are associated with said consensus region.

22. A method comprising forwarding a result obtained from the method of claim 15 to a remote location.

23. A method comprising transmitting data representing a result obtained from the method of claim 15 to a remote location.

24. A method comprising receiving a result obtained from a method of claim 15 from a remote location.

25. A system for identifying and selecting nucleic acid probes for detecting a target with a probe array, said system comprising:
means for selecting a plurality of candidate probes;
means for forming a plurality of clusters from said plurality of candidate probes according to hybridization characteristics of said candidate probes to a target sequence;
means for forming at least one SuperCluster from said clusters; and
means for selecting at least one probe from each said SuperCluster for said probe array.

26. The system of claim 25, further comprising means for identifying outlier probes not associated with any of said clusters.

27. The system of claim 25, further comprising means for identifying outlier probes each associated with one of said clusters, based on a metric different from a metric used for said forming a plurality of clusters.

28. The system of claim 27, wherein said metric different from a metric used for said forming a plurality of clusters comprises Euclidean distance measurement.

29. The system of claim 25, further comprising means for identifying SuperCluster outliers not associated with any of said SuperClusters.

30. The system of claim 25, further comprising:
means for compiling and aligning a plurality of nucleic acid transcripts to identify any sequence redundancy in said transcripts; and
means for identifying a consensus region for said plurality of transcripts.

31. A computer readable medium carrying one or more sequences of instructions for identifying and selecting nucleic acid probes for detecting a target with a probe array, wherein execution of one or more sequences of instructions by one or more processors causes the one or more processors to perform the steps of:
selecting a plurality of candidate probes;
forming a plurality of clusters from said plurality of candidate probes according to hybridization characteristics of said candidate probes to a target sequence;
forming at least one SuperCluster from said clusters; and
selecting at least one probe from each said SuperCluster for said probe array.

32. A computer readable medium carrying one or more sequences of instructions for identifying and selecting nucleic acid probes for detecting a target with a probe array, wherein execution of one or more sequences of instructions by one or more processors causes the one or more processors to perform the steps of:
selecting a plurality of candidate probes from a consensus region associated with a plurality of nucleic acid transcripts;
hybridizing nucleic acids from each of a plurality of tissue samples to each of a plurality of microarrays, each of said microarrays comprising said plurality of candidate probes;
forming a plurality of clusters from said plurality of probes according to hybridization characteristics of said candidate probes across said different tissue samples;
forming at least one SuperCluster from said clusters; and
selecting at least one probe from each said SuperCluster for said probe array.
